(19) United States
(12) Patent Application Publication
(10) Pub. No.: US 2007/0173732 A1
(43) Pub. Date: Jul. 26, 2007

(54) Method and apparatus for wireless brain interface

(76) Inventors: Elvir Causevic, New York, NY (US); George L. Engle, Maryville, IL (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 10/587,806

(22) PCT Filed: Jan. 28, 2005
(86) PCT No.: PCT/US05/03397

§ 371 Date: Jul. 27, 2006

Related U.S. Application Data
(60) Provisional application No. 60/540,288, filed on Jan. 29, 2004.

(51) Int. Cl. A61B 5/04

(52) U.S. Cl. 600/544; 128/903

(57) ABSTRACT

An implantable logic circuit configured to receive analog bioelectric signals from one or more implanted electrodes, perform amplification, A/D conversion of the received analog bioelectric signals, signal sampling, and to communicate the signals to a remote processing system over a wireless communications link. Power for the implantable logic circuit is derived from an external source over a wireless link.

### CROSS-REFERENCE TO RELATED APPLICATIONS

The present application is related to, and claims priority from, U.S. Provisional Application No. 60/540,288 filed on Jan. 29, 2004, herein incorporated by reference.

### TECHNICAL FIELD

The present invention is related generally to methods and apparatus for acquiring brainwave data from a living organism, and in particular, to a method and apparatus for utilizing a wireless link to provide power to, and acquire digital brainwave data from, an implanted data acquisition device within a living organism.

### BACKGROUND ART

A recently developed technology having widespread application in the field of data management and data acquisition is the use of Radio Frequency Identification (RFID) transponders or tags, which are a form of Automatic Identification and Date Capture (AIDC) technology, sometimes referred to as Automatic Data Capture (ADC) technology. The essence of AIDC technology is the ability to carry data in a suitable carrier and recover that data (read) or modify (write) it when required through a non-contact electromagnetic communications process across what is essentially an air interface.

AIDC utilizes wireless radio communications to uniquely identify objects by communicating with an AIDC transponder or tag associated with the object and programmed with unique identifying data related to an object or component. One type of AIDC transponder or tag consists of a logic circuit, a semiconductor memory, and a radio-frequency antenna configured to receive and transmit data. Numerous types and configurations of AIDC transponders or tags are known.

Data previously stored in the memory of the AIDC transponder or tag is optionally read or modified remotely over a wireless radio communications link, i.e. an air interface, to the AIDC transponder or tag, thereby providing features and capabilities not present with traditional bar code data storage. An AIDC interrogator containing a radio frequency transmitter-receiver unit used to query an AIDC transponder or tag. The AIDC interrogator optionally is disposed at a distance from the AIDC transponder or tag, and moving relative thereto. The AIDC transponder or tag detects the interrogating signal and transmits a response signal preferably containing encoded data stored in the semiconductor memory back to the interrogator. Such AIDC transponders or tags may have a memory capacity of 16 bytes to more than 64 kilobytes. In addition, the data stored in the AIDC transponder or tag semiconductor memory may optionally be re-written with new data or supplemented additional data transmitted from the AIDC interrogator.

Power for these data storage and logic circuits optionally is derived from an interrogating radio-frequency beam or from another external power source. Power for the transmission of data can also be derived from the RF beam or taken from another power source. As described in U.S. Pat. No. 6,107,910 to Nysen, and in the publication “Understanding RFID” by Prof. Anthony Furness, a variety of AIDC transponders or tags are known, such as surface acoustic wave devices, all of which provide power delivery, data storage, and data retrieval capabilities.

One field which can benefit greatly from improvements in wireless data acquisition is the field of biological signal and data acquisition. In particular, current systems for acquiring continuous or evoked bioelectric signals such as brain wave data from organisms typically rely upon a set of implanted electrodes or skin-contact electrodes which deliver electrical signals to a processing system consisting of signal amplification circuits, analog-to-digital conversion circuits, filter circuits, and eventually, to signal processing components wherein acquired brainwave signals are processed and evaluated.

These signal processing components are disposed external to the organism, and coupled to the implanted electrodes via cables or other suitable electrical conductors. The cable connectors linking the implanted electrodes with the processing system and any associated data storage systems significantly impact upon the normal activities of the organism. For example, when a small organism such as a mouse or rat is linked to such a system, the range of movement of the organism may be significantly limited by the length of cable. Correspondingly, when a larger, and potentially more inquisitive organism, such as a monkey, is linked to such a system, the risk of damage or disconnection of the cables from either the implanted electrodes or the processing system greatly increases.

Accordingly, it would be advantageous to provide an implantable data acquisition device configured to acquire brainwave signals from a living organism, and which is capable of utilizing a wireless interface to receive operating power and to communicate acquired data to an external processing system which is remotely disposed from the organism, enabling long-term acquisition of brainwave signals without the need for a physical connection to the external processing system.

### SUMMARY OF THE INVENTION

Briefly stated, an embodiment of the present invention provides an implantable logic circuit configured to receive analog bioelectric signals, perform A/D conversion of the received analog bioelectric signals, signal sampling, and to communicate the signals to a remote processing system over a wireless communications link. In the preferred embodiment, power for the implantable logic circuit is derived from an external source over a wireless link.

In an alternate embodiment of the present invention, the implantable logic circuit is implemented on a very large scale integrated architecture (VLSI).

In an alternate embodiment of the present invention, the implantable logic circuit includes signal amplification components for amplifying received analog bioelectric signals and one-bit sigma-delta sampling components for facilitating the A/D conversion of the received analog bioelectric signals.

In an alternate embodiment of the present invention, the implantable logic circuit is a component in a biological organism data acquisition system which includes a containment cage, an electrical winding disposed in proximity to the containment cage, the implantable logic circuit, and an external processing system operatively coupled to the implantable logic circuit via a wireless interface and configured to control the flow of electrical power to the implantable logic circuit through the electrical winding. The implantable logic circuit is configured for implantation into a living organism, such as a mouse, rat, or other small vertebrate, and is coupled to receive continuous or evoked analog bioelectric signals, such as brainwave signals from implantable electrodes also disposed within the living organism. When the organism is placed within the containment cage, the implantable logic circuit receives power via radio-frequency emissions from the electrical winding, and is configured to preprocess analog signals received via the implantable electrodes prior to wirelessly transmitting data to a receiver operatively coupled to the external processing system. The implantable logic circuit preprocesses the analog signals by first amplifying the received signals, performing an A/D conversion, and then utilizing a 1-bit sigma/delta sampling process to generate an output signal for wireless transmission to the external processing system.

In an alternate embodiment of the present invention, the implantable logic circuit is a component in a human patient brain activity monitoring system which includes an electrical winding disposed in proximity to a patient's head, the implantable logic circuit, and an external processing system operatively coupled to the implantable logic circuit via a wireless interface and configured to control the flow of electrical power to the implantable logic circuit through the electrical winding. The implantable logic circuit is configured for implantation into the human patient, and is coupled to receive analog bioelectric signals, such as continuous or evoked brainwave signals from implantable electrodes also disposed within the human patient. When the human patient is in proximity to the electrical winding, the implantable logic circuit receives power via radio-frequency emissions from the electrical winding, and is configured to preprocess analog signals received via the implantable electrodes prior to wirelessly transmitting data to a receiver operatively coupled to the external processing system. The implantable logic circuit preprocesses the analog signals by first amplifying the received signals, performing an A/D conversion, and then utilizing a 1-bit sigma/delta sampling process to generate an output signal for wireless transmission to the external processing system.

The foregoing and other objects, features, and advantages of the invention as well as presently preferred embodiments thereof will become more apparent from the reading of the following description in connection with the accompanying drawings.

### BRIEF DESCRIPTION OF THE DRAWINGS

In the accompanying drawings which form part of the specification:

FIG. 1 is a processing flow chart identifying steps carried out on the implantable logic circuit, and steps carried out in an external processing system;

FIG. 2 is a graphical representation of 1-bit sigma/delta sampling of a signal;

FIG. 3 is a representative layout of the implantable logic circuit; and

FIG. 4 is a simplified illustration of a data acquisition system of the present invention including a containment cage for a living organism.

Corresponding reference numerals indicate corresponding parts throughout the several figures of the drawings.

### BEST MODES FOR CARRYING OUT THE INVENTION

The following detailed description illustrates the invention by way of example and not by way of limitation. The description clearly enables one skilled in the art to make and use the invention, describes several embodiments, adaptations, variations, alternatives, and uses of the invention, including what is presently believed to be the best mode of carrying out the invention.

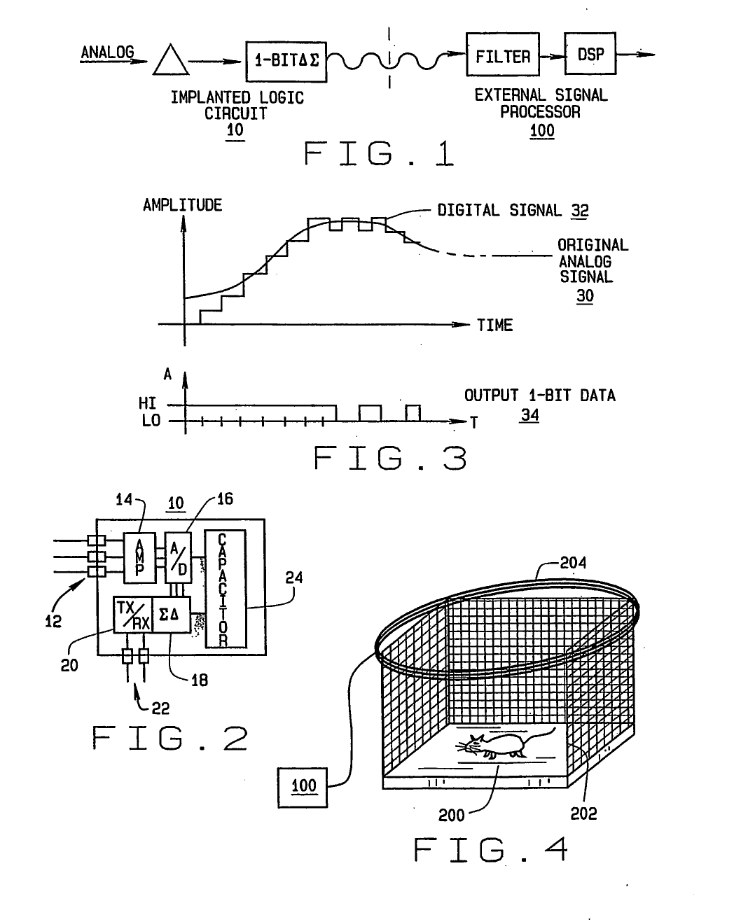

Turning to FIG. 1, the present invention provides an implantable logic circuit or signal processor 10 which is configured to perform the functions of receiving analog bioelectric signals, analog-to-digital conversion of the received analog bioelectric signals, signal sampling, and wireless communication of the sampled signals to an externally disposed remote processing system 100. In the preferred embodiment, power for the implantable logic circuit 10 is derived from an external source over an air interface or wireless link such as an electromagnetic field.

Preferably, as shown in FIG. 2, the implantable logic circuit 10 is implemented on a single integrated circuit, utilizing very large scale integrated (VLSI) circuit architecture, however, those of ordinary skill in the art will recognize that a wide variety of logic circuit architectures may be employed to build an implantable logic circuit having the desired functionality of the present invention. The implantable logic circuit 10 is encased in a matrix suitable for implantation in a living organism, and includes an input interface 12 through which analog signals from one or more implantable electrodes are received. Signals received at the interface 12 are passed to an amplifier circuit 14 and converted to digital format in an analog-to-digital converter circuit 16. The resulting digital signals are then routed to a sampling circuit 18 and conveyed to a transceiver circuit 20 for communication via a wireless interface 22 to the external signal processor 100. Power for the amplifier circuit 14, A/D converter circuit 16, sampling circuit 18, and transceiver circuit 20 is stored in a capacitor circuit 24, which includes an integrated antenna for receiving wireless power transmissions from an external source.

As shown in FIG. 3, the signal sampling carried by the sampling circuit 18 out on the implantable logic circuit 10 requires that an original analog signal 30 received through the implantable logic circuit input interface 12 be amplified at circuit 14 and converted to a digital signal 32 in the A/D converter circuit 16. Next, using 1-bit sigma-delta (Σ-Δ) sampling, the digital signal is converted into a 1-bit data stream 34 by the sampling circuit 18, wherein a “1” or high signal indicates an increase in signal amplitude, and a “0” or low signal indicated a decrease in signal amplitude. The resulting 1-bit data stream 34 is communicated via the wireless communications link 22 to the external signal processor 100, where it is filtered and processed as required, depending upon the particular type of brain activity signal. Processing is preferably performed in the external signal processor 100 to maintain the power consumption of the implantable logic circuit 10 at a reduced level which can be adequately supplied via the wireless link.

Those of ordinary skill in the art will recognize that a variety of signal sampling methods may be implemented within the scope of the present invention, and that the subsequent processing of the resulting data stream by the external signal processor 100 is highly dependant upon the particular type of brain activity signal being processed, and on the type of data which the system is acquiring.

As shown in FIG. 4, the implantable logic circuit 10 of the present invention may be utilized to acquire data from a living organism 200, such as a mouse, rat, or other vertebrate animal in a minimally invasive manner over an extended period of time. With the logic circuit 10 surgically implanted within the organism 200, and operatively coupled to similarly implanted sampling electrodes, analog bioelectric signals received through the implanted electrodes can be monitored by the external system 100 without requiring the organism 200 to be restrained or coupled to an electrical connection. Preferably, for small organisms such as mice, rats, rabbits, etc., the organism 200 is contained within a containment cage 202, and an electrical winding 204 is disposed in proximity to the containment cage 202. For larger organisms, such as a human patient, the electrical winding 204 may be disposed in wearable article, such as a headband, or disposed in proximity to the patient's head by incorporation into an examination chair or surgical table.

The implantable logic circuit 10 and external processing system 100 are operatively coupled via the wireless interface 22. The external processing system 100 is further configured to control a wireless flow of electrical power to the implantable logic circuit 10 through an electromagnetic field generated by a controlled flow of electrical current through the electrical winding 204.

When the organism 200 is placed within the containment cage 202, or in proximity to the electrical winding 204, the implantable logic circuit 10 receives power via radio-frequency emissions from the electrical winding 204, and is configured to process the analog signals 30 received via the implantable electrodes prior to wirelessly transmitting a data stream 34 to a receiver associated with the external processing system 100. The implantable logic circuit 10 preferably processes the analog signals 30 by first amplifying the received signals, performing an A/D conversion, and then utilizing a 1-bit sigma/delta sampling process to generate an output data stream 34 for wireless transmission to the external processing system 100.

In an alternate embodiment, the implantable logic circuit 10 of the present invention may be utilized to acquire data from a human patient in a minimally invasive manner over an extended period of time, or as part of a brain-state monitoring system. For example, as part of an anesthesia and sedation monitoring system which provides an index representative of a human patient's level of anesthesia or sedation by monitoring one or more evoked bio-potential signals and/or random electroencephalogram activity to observe changes over time in response to the administration of an anesthetic or sedative. An exemplary brain-state/depth of anesthesia and sedation monitoring system is described in co-pending U.S. patent application Ser. No. 10/485,750, published as Patent Application Publication No. US 2004/0243017 A1, herein incorporated by reference.

With the logic circuit 10 surgically implanted within human patient, and operatively coupled to similarly implanted electrodes, analog bioelectric signals received through the implanted electrodes can be monitored by the external system 100 without requiring the human patient to be restrained or coupled to the external system 100 with an electrical connection. The external system 100 may be configured as a wearable unit, maintained in proximity to the human patient, or as a stationary unit, for example, maintained in a doctor's office or surgical suite, which is utilized at intervals to acquire brain activity information from the human patient.

When in proximity, the implantable logic circuit 10 and external processing system 100 are operatively coupled via the wireless interface 22. The external processing system 100 is further configured to control a wireless flow of electrical power to the implantable logic circuit 10 through the electrical winding 204.

The implantable logic circuit 10 receives power via radio-frequency emissions from the electrical winding 204. The received power is utilized in the logic circuit 10 to process the analog signals 30 received via the implantable electrodes prior to wirelessly transmitting a data stream 34 to a receiver associated with the external processing system 100. The implantable logic circuit 10 preferably processes the analog signals 30 by amplifying the received signals, performing an A/D conversion, and then utilizing a 1-bit sigma/delta sampling process to generate an output data stream 34 for wireless transmission to the external processing system 100. Subsequent processing of the data stream 34 is performed in a conventional manner by the external processing system 100, reducing the power requirements for the implantable logic circuit 10.

Those of ordinary skill in the art will recognize that the embodiments of the present invention described herein are particularly suited to provide a means for monitoring the brainwave activity of an organism 200, but may be readily adapted to provide a means for monitoring other bioelectric signals in the organism 200 merely by suitable placement of the implantable electrodes which are coupled to the implantable logic circuit 10.

The present invention can be embodied in-part the form of computer-implemented processes and apparatuses for practicing those processes. The present invention can also be embodied in-part the form of computer program code containing instructions embodied in tangible media, such as floppy diskettes, CD-ROMs, hard drives, or an other computer readable storage medium, wherein, when the computer program code is loaded into, and executed by, an electronic device such as a computer, micro-processor or logic circuit, the device becomes an apparatus for practicing the invention.

The present invention can also be embodied in-part the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented in a general-purpose microprocessor, the computer program code segments configure the microprocessor to create specific logic circuits.

In view of the above, it will be seen that the several objects of the invention are achieved and other advantageous results are obtained. As various changes could be made in the above constructions without departing from the scope of the invention, it is intended that all matter contained in the above description or shown in the accompanying drawings shall be interpreted as illustrative and not in a limiting sense.

1. An implantable bioelectric signal processing system comprising:
a logic circuit adapted for implantation within a living organism, said logic circuit configured to perform analog to digital conversions of received analog bioelectric signals;
wherein said logic circuit includes an interface configured to receive an analog bioelectric signal from at least one electrode implanted in said living organism;
a signal sampling circuit operatively coupled to receive said analog signal from said interface, said sampling circuit configured to generate a 1-bit output signal associated with said analog signal; and
a transceiver coupled to said sampling circuit, said transceiver configured to communicate said output signal from said signal sampling circuit to a remote processing system over a wireless communications link.

2. The implantable bioelectric signal processing system of claim 1 wherein said interface, said sampling circuit, and said transceiver are disposed within a common matrix configured for implantation within an organism.

3. The implantable bioelectric signal processing system of claim 1 further including a power distribution means configured to receive electrical power from a remote power source over a wireless link.

4. The implantable bioelectric signal processing system of claim 1 further including a capacitor circuit operatively coupled to an integrated antenna for receiving wireless power transmissions from an external power source.

5. The implantable bioelectric signal processing system of claim 1 wherein said sampling circuit is configured for 1-bit sigma/delta sampling of said received signals.

6. The implantable bioelectric signal processing system of claim 1 wherein said interface, said signal sampling circuit, and said transceiver are implemented on a single integrated circuit.

7. The implantable bioelectric signal processing system of claim 6 wherein said single integrated circuit utilizes Very Large Scale Integrated circuit architecture.

8. The implantable bioelectric signal processing system of claim 1 wherein said interface includes a signal amplification component for amplifying said received analog bioelectric signal.

9. A biological organism data acquisition system including:
an implantable logic circuit configured for implantation in an organism;
an electrical winding disposed in proximity to said organism, said electrical winding configured to receive a controlled flow of electrical current from an electrical power source and to generate an electromagnetic field;
an external signal processing system operatively coupled to said implantable logic circuit via a wireless interface, said external signal processing system configured to control a flow of electrical power to said implantable logic circuit through said electrical winding and generated electromagnetic field via an air interface;
wherein responsive to said flow of electrical power via said air interface, said implantable logic circuit is coupled to receive analog bioelectric signals from said organism through at least one implantable electrode disposed within said organism, and to communicate data associated with said analog bioelectric signals to said external signal processing system via a wireless communications link.

10. The biological organism data acquisition system of claim 9 wherein said implantable logic circuit is further configured to process said received analog signals to obtain said associated data for wireless communication to said external processing system.

11. The biological organism data acquisition system of claim 10 wherein said implantable logic circuit is further configured to amplify said received analog signals, to convert said received analog signals to digital signals with a 1-bit sigma/delta sampling process to obtain said associated data for wireless communication to said external processing system.

12. A biological organism data acquisition system including:
an implantable logic circuit configured for implantation in an organism;
an electrical winding disposed in proximity to said organism, said electrical winding configured to receive a controlled flow of electrical current from an electrical Dower source and to generate an electromagnetic field;
an external signal processing system operatively coupled to said implantable logic circuit via a wireless interface, said external signal processing system configured to control a flow of electrical power to said implantable logic circuit through said electrical winding and generated electromagnetic field via an air interface;
wherein said implantable logic circuit is coupled to receive analog bioelectric signals from said organism through at least one implantable electrode disposed within said organism, and to communicate data associated with said analog bioelectric signals to said external signal processing system via a wireless communications link; and
further including an organism containment cage, said electrical winding disposed in proximity to said organism containment cage to generate an electromagnetic field within said organism containment responsive to said controlled flow of electrical current from said electrical power source.

13. A method for acquiring bio-electric signals from an organism, the method comprising the steps of:
implanting at least one electrode in the organism, said electrode configured to acquire at least one bio-electric signal;
implanting a logic circuit in the organism, said implanted logic circuit coupled to said electrode to receive said acquired bio-electric signal;
providing electrical power to said implanted logic circuit from an external power source;
receiving, responsive to said provided power, said at least one bio-electric signal at said logic circuit from said implanted electrode; and
communicating data representative of said received bio-electric signal from said implanted logic circuit to an external data processor via a wireless communications link.

14. The method of claim 13 further including the step of converting said received bio-electric signals from analog to digital form in said implanted logic circuit, prior to said communicating step.

15. The method of claim 14 wherein said step of converting includes processing said received bio-electric signal through a sigma-delta analog to digital converter.

16. The method of claim 13 wherein said at least one electrode is implanted to acquire at least one brain activity bio-electric signal in the organism.

17. The method of claim 13 wherein said received bio-electric signal is a continuous bio-electric signal.

18. The method of claim 13 wherein said received bio-electric signal is an evoked bio-electric signal.

19. The method of claim 13 wherein said step of providing electrical power to said implanted logic circuit includes transferring electrical power from said external power source to said implanted logic circuit over a wireless interface.

20. The method of claim 13 wherein said step of providing electrical power to said implanted logic circuit includes generating an electro-magnetic field with said external power source;
disposing said implanted logic circuit within said electromagnetic field; and
extracting electrical power from said electro-magnetic field at said implanted logic circuit.
